(19) United States
(12) Patent Application Publication
(10) Pub. No.: US 2005/0020637 A1
(43) Pub. Date: Jan. 27, 2005

(54) Agents for the treatment of airway disorders

(76) Inventor: Wolfgang-Alexander Simon, Konstanz (DE)

Correspondence Address: NATH & ASSOCIATES PLLC, 1030 FIFTEENTH STREET, N.W., SIXTH FLOOR, WASHINGTON, DC, 20005, US

(21) Appl. No.: 10/495,804

(22) PCT Filed: Nov. 16, 2002
(86) PCT No.: PCT/EP02/12864

(30) Foreign Application Priority Data
Nov. 19, 2001 (EP) 01000642.7

(51) Int. Cl. A61K 31/4439

(52) U.S. Cl. 514/338

(57) ABSTRACT

The invention relates to the use of reversible proton pump inhibitors in the treatment of airway disorders.

### TECHNICAL FIELD

The invention relates to the use of compounds from the class consisting of the acid secretion inhibitors for the treatment of airway disorders.

### PRIOR ART

A whole series of compounds are known from the prior art which inhibit gastric acid secretion by reversible blockade of the proton pump and which have therefore also been designated as “reversible proton pump inhibitors” (RPPI) or recently as “APAs” (acid pump antagonists). These compounds should be suitable for the treatment of gastric and intestinal disorders.

### DESCRIPTION OF THE INVENTION

Surprisingly, it has now been found that the reversible proton pump inhibitors, whose original field of use is the treatment of gastric and intestinal disorders, are particularly suitable for the treatment of airway disorders.

The invention thus relates in a first aspect to the use of reversible proton pump inhibitors in the treatment of airway disorders including bronchoconstriction.

Reversible proton pump inhibitors are designated as those substances which inhibit gastric acid secretion by blockade of the proton pump, but which, in contrast to the PPIs, do not bind covalently to the H⁺/K⁺-ATPase, the enzyme responsible for gastric acid secretion. The term “reversible proton pump inhibitor” according to the invention comprises not only the active compounds as such, but also their pharmacologically tolerable salts and solvates (in particular hydrates) etc.

Reversible proton pump inhibitors are described and claimed, for example, in the following patent applications and patents: EP 33094, EP 204285, EP 228006, EP 233760, EP 259174, EP 266326, EP 266890, EP 270091, EP 307078, EP 308917, EP 330485, U.S. Pat. No. 4,728,658, U.S. Pat. No. 5,362,743, WO 9212969, WO 9414795, WO 9418199, WO 9429274, WO 9510518, WO 9527714, WO 9603405, WO 9604251, WO 9605177, WO 9703074, WO 9703076, WO 9747603, WO 9837080, WO 9842707, WO 9843968, WO 9854188, WO 9909029, WO 9928322, WO 9950237, WO 9951584, WO 9955705, WO 9955706, WO 0001696, WO 0010999, WO 0011000, WO 0017200, WO 0026217, WO 0029403, WO 0063211, WO 0077003, WO 0158901, WO 0172754, WO 0172755, WO 0172756, WO 0172757, WO 02034749, WO 02060440, WO 02060441 and WO 02060442.

Examples of reversible proton pump inhibitors which can be mentioned on the basis of their (proposed) INNs or their code name are the compounds: AG-2000 (EP 233760), AU461 (WO 9909029), BY112 (WO 9842707), Soraprazan (BY359) (WO 0017200), CP-113411 (U.S. Pat. No. 5,362,743), DBM-819 (WO 0001696), KR-60436 (WO 9909029), Pumaprazole (WO 9418199), SKF-96067 (EP 259174), SKF-96356 (EP 307078), SKF-97574 (EP 330485), T-330 (EP 270091), T-776 (EP 270091), WY-27198 (U.S. Pat. No. 4,728,658), YH-1885 (WO 9605177), YJA-20379-8 (NO 9703074), YM-19020 (EP 266890) and 2,3-dimethyl-8-(2-ethyl-6-methylbenzylaminoyimidazo(1,2-a)pyridine-6-carboxamide (e.g. WO 02060440).

Of these, the compounds AU461, Soraprazan (BY359), DBM-819, KR-60436, T-330, YH-1885, YJA-203798 and 2,3-dimethyl-8-(2-ethyl-6-methylbenzylamino)-imidazo(1,2-a)pyridine-6-carboxamide are particularly worthy of mention.

A particularly interesting group of reversible proton pump inhibitors is described and claimed in international patent applications WO 9842707, WO 9854188, WO 0017200, WO 0026217, WO 0063211, WO 0172754, WO 0172755, WO 0172756, WO 0172757 and WO 02034749.

As examples of compounds to be used according to the invention, the following can be mentioned:
(7S,8R,9R)-2,3-dimethyl-7,8-dihydroxy-9-phenyl-7,8,9,10-tetrahydroimidazo[1,2-h][1,7]naphthyridine,
(7R,8R,9R)-3-hydroxymethyl-7,8-dihydroxy-2-methyl-9-phenyl-7,8,9,10-tetrahydroimidazo[1,2-h][1,7]naphthyridine,
(7S,8R,9R)-7,8-isopropylidenedioxy-2,3-dimethyl-9-phenyl-7,8,9,10-tetrahydroimidazo[1,2-h][1,7]naphthyridine,
7,8-dihydroxy-9-phenyl-2,3-dimethyl-7H-8,9-dihydropyrano[2,3-c]imidazo[1,2-a]pyridine,
(7R,8R,9R)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8R,9R)-2,3-dimethyl-hydroxy-7-methoxy-9-phenyl-7,8,9,10-tetrahydroimidazo[1,2-h][1,7]naphthyridine,
(7R,8S,9S)-2,3-dimethyl-8-hydroxy-7-methoxy-9-phenyl-7,8,9,10-tetrahydroimidazo[1,2-h][1,7]naphthyridine,
(7R,8R,9R)-2,3-dimethyl-7-ethoxy-8-hydroxy-9-phenyl-7,8,9, 1-tetrahydroimidazo[1,2-h][1,7]naphthyridine,
(7S,8R,9R)-2,3-dimethyl-7-ethoxy-8-hydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7,8,9,10-tetrahydroimidazo[1,2-h][1,7]naphthyridine,
(7S,8S,9S)2,3-dimethyl-8-hydroxy-7-(2-methoxyethoxy)-9-phenyl-7,8,9,10-tetrahydrolmidazo[1,2-h][1,7]naphthyridine,
(7S,8R,9R)-2,3-dimethyl-8-hydroxy-7-(2-methoxyethoxy)-9-phenyl-7,8,9,10-tetrahydroimidazo[1,2-h][1,7]naphthyridine,
(7R,8S,9S)-2,3-dimethyl-8-hydroxy-7-(2-methoxyethoxy)-9-phenyl-7,89,10-tetrahydroimidazo[1,2-h][1,7]naphthyridine,
(7S,8R,9R)-2,3-dimethyl-8-hydroxy-9-phenyl-7-(2-propoxy)-7,8,9,10-tetrahydroimidazo[1,2-h][1,7]naphthyridine,
(7R,8R,9R)-2,3-dimethyl-7,8-dimethoxy-9-phenyl-7,8,9,10-tetrahydroimidazo[1,2-h][1,7]naphthyridine,
(7R,8R,9R)-2,3-dimethyl-8-hydroxy-7-(2-methylthioethyloxy)-9-phen-7,8,9,10-tetrahydroimidazo[1,2-h][1,7]naphthyridine,
(7S,8R,9R)-2,3-dimethyl-8-hydroxy-7-(2-methylthioethyloxy)-9-phenyl-7,8,9,10-tetrahydroimidazo[1,2-h][1,7]naphthyridine,
(7R,8R,9R)2,3-dimethyl-B-hydroxy-7-(2-methylsulphinylethoxy)-9-phenyl-7,8,9,10-tetrahydroimidazo[1,2-h][1,7]naphthyridine,
(7S,8R,9R)-2,3-dimethyl-8-hydroxy-7-(2-methylsulphinylethoxy)-9-phenyl-7,8,9,10-tetrahydroimidazo[1,2-h][1,7]naphthyridine,
(7R,8R,9R)-2,3-dimethyl-8-hydroxy-7-(ethylthio)-9-phenyl-7,8,9,10-tetrahydroimidazo[1,2-h][1,7]naphthyridine,
(7S,8R,9R)-2,3-dimethyl-8-hydroxy-7-(ethylthio)-9-phenyl-7,8,9,10-tetrahydroimidazo[1,2-h][1,7]naphthyridine,
(7R,8R,9R)-2,3-dimethyl-8-hydroxy-7-(2,2,2-trifluoroethoxy)-9-phenyl-7,8,9,10-tetrahydroimidezo[1,2-h][1,7]naphthyridine,
(7S,8R,9R)-2,3-dimethyl-8-hydroxy-7-(2,2,2-trifluoroethoxy)-9-phenyl-7,8,9,10-tetrahydroimidazo[1,2-h][1,7]naphthyridine,
(7S,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-methoxy-2,3-dimethyl-9-phenyl-7,8,9,10-tetrahydroimidazo[1,2-h][1,7]naphthyridine,
(7R,8R,9R)-8-acetoxy-7-ethoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-9-phenyl-8-propionyloxy-7,8,9,10-tetrahydromidazo[1,2-h][1,7]naphthyridine,
(7R,8R,9R)-8-benzoyloxy-7-(2-methoxyethoxy)-2,3-dimethyl-9-phenyl-7,8,9,10-tetrahydroimidazo[1,2-h][1,7]naphthyridine,
(7S,8R,9R)-8-benzoyioxy-72-methoxyethoxy)2,3-dimethyl-9-phenyl-7,8,9,10-tetrahydroimidazo[1,2-h][1,7]naphthyridine,
(7R,8R,9R)-8-methoxycarbonyloxy-7-(2-methoxyethoxy)-2,3-dimethyl-9-phenyl-7,89,10-tetrahydroimidazo[1,2-h][1,7]naphthyridine,
(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-methoxy-2,3-dimethyl-9-phenyl-7,8,9,10-tetrahydroimidazo[1,2-h][1,7]naphthyridine,
(7S,8R,9R)-8-benzoyloxy-7-methoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7S,8R,9R)-7-methoxy-2,3-dimethyl-8-(3-nitrobenzoyloxy)-9-phenyl-7,8,9,10-tetrahydroimidazo[1,2-h][1,7]naphthyridine,
(7R,8R,9R)-7-methoxy-2,3-dimethyl-8-(3-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethy-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-2,3-dimethyl-8-(N,N-dimethylaminomethylcarbonyoxy)-9-phenyl-7,8,9,10-tetrahydroimidazo[1,2-h][1,7]naphthyridine,
(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-8-ethylaminocarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-2,3-dimethyl-7-(2-methoxyethoxy)-9-phenyl-7H-8,9-dihydropyrano[2,3-c]imidazo[1,2-a]pyridine,
(7S,8R,9R)-8-benzoyloxy-2,3-dimethyl-7-(2-methoxyethoxy)-9-phenyl-7H-8,9-dihydropyrano[2,3-c]imldazo[1,2-a]pyridine,
(7R,8R,9R)-8-[4-(methoxycarbonyl)-benzoyloxy]2,3-dimethyl-7-(2-methoxyethoxy)-9-phenyl-7H-8,9-dihydropyrano[2,3-c]imidazo[1,2-a]pyridine,
(7S,8R,9R)-B-[4-(methoxycerbanyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-2.3-dimethyl-7-methoxy-8-methoxyacetyloxy-9-phenyl-7.8.9.10-tetrahydroimidazo[1.2-h][1.7]naphthyridine,
(7R,8R,9R)-8-(N,N-diethylaminocarbonyloxy)-2.3-dimethyl-7-methoxy-9-phenyl-7.8.9.10-tetrahydroimidazo[1.2-h][1.7]naphthyridine,
(7S,8R,9R)-8-(N,N-diethylaminocarbonyloxy)-2.3-dimethyl-7-methoxy-9-phenyl-7.8.9.10-tetrahydroimidazo[1.2-h][1.7]naphthyridine,
(7R,8R,9R)-7-methoxy-8-methoxycarbonyloxy-2.3-dimethyl-9-phenyl-7.8.9.10-tetrahydroimidazo[1.2-h][1.7]naphthyridine,
(7S,8R,9R)-7-methoxy-8-methoxycarbonyloxy-2.3-dimethyl-9-phenyl-7.8.9.10-tetrahydroimidazo[1.2-h][1.7]naphthyridine,
(7R,8R,9R)-2.3-dimethyl-8-formyloxy-7-methoxy-9-phenyl-7.8.9.10-tetrahydrolmidazo[1.2-h][1.7]naphthyridine,
(7S,8R,9R)-2.3-dimethyl-8-formyloxy-7-methoxy-9-phenyl-7.8.9.10-tetrahydroimidazo[1.2-h][1.7]naphthyridine,
(7R,8R,9R)-8-benzoyloxy-2.3-dimethyl-7-methoxy-9-phenyl-7.8.9.10-tetrahydroimidazo[1.2-h][1.7]naphthyridine,
(7R,8S,9R)-2,3,8-trimethyl-7,8-dihydroxy-9-phenyl-7,8,9,10-tetrahydroimidazo[1,2-h][1,7]naphthyridine,
(7S,8S,9R)-2,3-dimethyl-8-benzyl-7,8-dihydroxy-9-phenyl-7,8,9,10-tetrahydroimidazo[1,2-h][1,7]-naphthyridine,
(7R,8S,9R)-2,3,8-trimethyl-7,8,0,0-isopropylidene-9-phenyl-7,8,9,10-tetrahydroimidazo[1,2-h]-[1,7]naphthyridine,
(7S,8S,9R)-2,3,8-trimethyl-7-(2-methoxyethoxy)-8-hydroxy-9-phenyl-7,8,9,10-tetrahydroimidazo[1,2-h][1,7]naphthyridine,
(7S,8S,9R)-2,3,8-trimethyl-7-methoxy-8-hydroxy-9-phenyl-7,8,9,10-tetrahydrolmidazo[1,2-h][1,7]-naphthyridine,
(7R,8R,9R)-2,3,7-trimethyl-7,8-dihydroxy-9-phenyl-7,8,9,10-tetrahydroimidazo[1,2-h][1,7]naphthyridine,
(7R,8R,9R)-2,3,7-trimethyl-7,8-[1,3]dioxolo-9-phenyl-7,8,9,10-tetrahydroimidazo[1,2-h][1,7]naphthyridine,
(8S,9R)-2,3-dimethyl-8-hydroxy-7-methylidene-9-phenyl-7,8,9,10-tetrahydrolmidazo[1,2-h][1,7]-naphthyridine,
(7S,8R,9R)-2,3,7-trimethyl-7,8-dihydroxy-9-phenyl-7H-8,9-dihydropyrano[2,3-c]imidazo[1,2-a]pyridine,
(7R,8R,9R)-2,3,7-trimethyl-7,8-dihydroxy-9-phenyl-7H-8,9-dihydropyrano[2,3-c]imidazo[1,2-a]pyridine,
(7S,8R,9R)-2,3-dimeth)-4-7,8-dihydroxy-7,9-diphenyl-7H-8,9-dihydropyrano[2,3-c]imidazo[1,2-a]pyridine,
(7S,8R,9R)-2,3-dimethyl-72′,2′-dimethylvinyl)-7,8-dihydroxy-9-phenyl-7H-8,9-dihydropyrano[2,3-c]imidazo[1,2-a]pyridine,
(7R,8R,9R)-2,3-dimethyl-7,8-O-isopropylidene-9-phenyl-7-vinyl-7H-8,9-dihydropyrano[2,3-c]-imidazo[1,2-a]pyridine,
(7R,8R,9R)-2,3-dimethyl-8-hydroxy-7-(2-methoxyethoxy)-9-phenyl-7H-8,9-dihydropyrano[2,3-c]-imidazo[1,2-a]pyridine,
(7S,8R,9R)-2,3-dimethyl-8-hydroxy-72-methoxyethoxyyg-phenyl-7H-8,9-dihydropyrano[2,3-c]imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]pyridine,
(7S,8R,9R)-2,3-dimethyl-8-hydroxy-7-ethoxy-phenyl-7H-8,9-dihydropyrano[2,3-c]imidazo[1,2-a]pyridine,
(7R,8R,9R)-2,3-dimethyl-8-hydroxy-7-(2-methoxypropoxy)-9-phenyl-7H-8,9-dihydropyrano[2,3-c]imidazo[1,2-a]pyridine,
(7S,8R,9R)-2,3-dimethyl-B-hydroxy-7(2-methoxypropoxy)-9-phenyl-7H-8,9-dihydropyrano[2,3-c]imidazo[1,2-a]pyridine,
(7R,8R,9R)-2,3-dimethyl-8-hydroxy-7-(2-propoxy)-9-phenyl-7H-8,9-dihydropyrano[2,3-c]imidezo[1,2-a]pyridine,
(7S,8R,9R)-2,3-dimethyl-8-hydroxy-7-(2-propoxy)-9-phenyl-7H-8,9-dihydropyrano[2,3-c]imidazo[1,2-a]pyridine,
(7R,8R,9R)-2,3-dimethyl-8-hydroxy-7-butoxy-9-phenyl-7H-8,9-dihydropyrano[2,3-c]imidazo[1,2-a]pyridine,
(7S,8R,9R)-2,3-dimethyl-8-hydroxy-7-butoxy-9-phenyl-7H-8,9-dihydropyrano[2,3-c]imidazo[1,2-a]pyridine,
(7S,8R,9R)-7,8-dihydroxy-6-methoxymethyl-2,3-dimethyl-9-phenyl-7,8,9,10-trahydroimidazo[1,2-h][1,7]naphthyridine,
(7R,8R,9R)-7,8-dihydroxy-6-methoxymethyl-2,3-dimethyl-9-phenyl-7,8,9,10-tetrahydroimidazo[1,2-h][1,7]naphthyridine,
(7S,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-methoxy-6-methoxymethy-2,3-dimethyl-9-phenyl-7,8,9,10-tetrahydroimidazo[1,2-h][1,7]naphthyridine,
(7R,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7S,1R,9R)-18-hydroxy-7-(2-methoxyethoxy)-6-methoxymethyl-2,3-dimethyl-9-phenyl-7,8,9,10-tetrahydroimidazo[1,2-h][1,7]naphthyridine,
(7R,8R,9R)-8-hydroxy-7-ethoxy-6-methoxymethyl-2,3-dimethyl-9-phenyl-7,8,9,10-tetrahydroimidazo[1,2-h][1,7]naphthyridine,
(7S,8R,9R)-4-hydroxy-7-ethoxy-6-methoxymethyl-2,3-dimethyl-9-phenyl-7,8,9,10-tetrahydroimidazo[1,2-h][1,7]naphthyridine,
7,8-dihydroxy-2,3-dimethyl-9-(3-thienyl)-7,8,9,10-tetrahydroimidazo[1,2-h][1,7]naphthyridine,
7-hydroxy-2,3-dimethyl-9-(3-thienyl)-7,8,9,10-tetrahydroimidazo[1,2-h][1,7]naphthyridine,
9-(3-furyl)-7-hydroxy-2,3-dimethyl-7,8,9,10-tetrahydroimidazo[1,2-h][1,7]naphthyridine,
(7R,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7S,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7R,8R,9R)-7,8-dihydroxy-2-methyl-9-phenyl-7,8,9,10-tetrahydroimidazo[1,2-h][1.7]naphthyridine,
(7S,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-8-hydroxy-2-methyl-7-(2-methoxyethoxy9-phenyl-7,8,9,10-tetrahydroimidazo[1,2-h][1,7]naphthyridine,
(7R,8R,9R)-3-bromo-8-hydroxy-7-(2-methoxyethoxy)-2-methyl-9-phenyl-7.8.9.10-tetrahydroimidazo[1.2-h][1.7]naphthyridine,
(7R,8R,9R)-3-chloro-8-hydroxy-7-(2-methoxyethoxy)-2-methyl-9-phenyl-7.8.9.10-tetrahydroimidazo[1.2-h][1.7]naphthyridine,
(7R,8R,9R)-3-bromo-7-hydroxy-8-(2-methoxyethoxy)-2-methyl-9-phenyl-7.8.9.10-tetrahydroimidazo[1.2-h][1.7]naphthyridine,
(7R,8R,9R)-3-chloro-8-hydroxy-7-(2-methoxyethoxy)-2-methyl-9-phenyl-7H-8,9-dihydro-pyrano[2,3-c]imidazo[1,2-a]pyridine,
(7R,8R,9R)-8-hydroxy-7-(2-methoxyethoxy)-2-methyl-9-phenyl-7H-8,9-dihydro-pyrano[2,3-c]imidazo[1,2-a]pyridine,
(7R,8R,9R)-7,8-dihydroxy-2-methyl-9-phenyl-7H-8,9-dihydropyrano[2,3-c]imidazo[1,2-a]pyridine,
(7S,8R,9R)-7,8-dihydroxy-2-methyl-9-phenyl-7.8.9.10-tetrahydroimidazo[1.2-h][1.7]naphthyridine,
(7R,8R,9R)-8-hydroxy-7-methoxy-2-methyl-9-phenyl-7.8.9.10-tetrahydroimidazo[1.2-h][1.7]naphthyridine,
(7S,8R,9R)-8-hydroxy-7-methoxy-2-methyl-9-phenyl-7.8.9.10-tetrahydroimidazo[1.2-h][1.7]naphthyridine,
(7R,8R,9R)-3-hydroxymethyl-8-hydroxy-7-(2-methoxyethoxy)-2-methyl-9-phenyl-7.8.9.10-tetrahydroimidazo[1.2-h][1.7]naphthyridine,
(7R,8R,9R)-3-hydroxymethyl-8-hydroxy-7-(2-hydroxyethoxy)-2-methy-9-phenyl-7.8.9.10-tetrahydroimidazo[1.2-h][1.7]naphthyridine,
(7R,8R,9R)-2,3-dimethyl-8-hydroxy-7-(2-hydroxyethoxy)-9-phenyl-7.8.9.10-tetrahydroimidazo[1.2-h][1.7]naphthyridine,
(7R,8R,9R)-3,9-diphenyl-8-hydroxy-7-(2-methoxyethoxy)-2-methyl-7.8.9.10-tetrahydroimidazo[1.2-h][1.7]naphthyridine,
(7R,8R,9R)-7,8-dihydroxy-2-methoxymethyl-3-methyl-9-phenyl-7,8,9,10-tetrahydroimidazo[1,2-h][1,7]naphthyridine,
(7S,8R,9R)-8-hydroxy-7-(2-methoxyethoxy)-2-methoxymethyl-3-methyl-9-phenyl-7,8,9,10-tetrahydro imidazo[1,2-h][1,7]naphthyridine,
(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7S,8R,9R)-7-ethoxy-8-hydroxy-2-methoxymethyl-3-methyl-9-phenyl-7,8,9,10-tetrahydroimidazo[1,2-h][1,7]naphthyridine,
(7R,8R,9R)-7-ethoxy-8-hydroxy-2-methoxymethyl-3-methyl-9-phenyl-7,8,9,10-tetrahydroimidazo[1,2-h][1,7]naphthyridine,
   and the pharmacologically compatible salts of these compounds.

An example of a preferred reversible proton pump inhibitor which may be mentioned is the compound (7R,8R,9R)-2,3-dimethyl-8-hydroxy-7-(2-methoxyethoxy)-9-phenyl-7,8,9,10-tetrahydroimidazo-[1,2-h][1,7]naphthyridine.

Airway disorders to be treated which may be mentioned in particular are pulmonary abnormalities such as bronchitis (including COPD), asthma (particularly night-time asthma attacks), pneumonitis and pulmonary fibrosis.

The invention relates in a further aspect to the use of reversible proton pump inhibitors for the treatment of patients who are suffering from an airway disorder.

The invention further relates to a method for the treatment of airway disorders which consists in administering to a patient who needs such a treatment an effective amount of a reversible proton pump inhibitor.

The invention further relates to the use of reversible proton pump inhibitors for the production of medicaments for the treatment of airway disorders.

The invention further relates to a pharmaceutical preparation for the treatment of airway disorders which contains a reversible proton pump inhibitor as active compound.

The invention further relates to a ready-to-use medicament, comprising a reversible proton pump inhibitor as active compound, which contains a reference to the fact that this ready-to-use medicament can be employed for the treatment of airway disorders.

Commercial Utility

According to the invention, the reversible proton pump inhibitors are employed for the treatment of airway disorders in the form of ready-to-use medicaments. These medicaments are prepared by methods known per se familiar to the person skilled in the art. As medicaments, the reversible proton pump inhibitors are either used here as such, or preferably in combination with suitable pharmaceutical excipients or vehicles in the form of tablets, coated tablets, capsules, suppositories, patches (e.g. as TTS), emulsions, suspensions or solutions, the active compound content advantageously being between 0.1 and 95% and it being possible by means of the appropriate choice of the excipients and vehicles to achieve a pharmaceutical administration form exactly adapted to the active compound and/or to the desired onset of action and/or to the duration of action (e.g. a sustained release form or an enteric form).

The person skilled in the art is familiar on the basis of his/her expert knowledge with which excipients or vehicles are suitable for the desired pharmaceutical formulations. Besides solvents, gel-forming agents, suppository bases, tablet excipients and other active compound carriers, it is possible to use, for example, antioxidants, dispersants, emulsifiers, antifoams, taste corrigents, preservatives, solubilizers, colorants or, in particular, permeation promoters and complexing agents (e.g. cyclodextrins).

The active compounds can be administered orally, parenterally or percutaneously.

In general, it has proved advantageous in human medicine to administer the reversible proton pump inhibitor in a daily dose of, in particular, 0.1 to 1.5 mg/kg of body weight, if appropriate in the form of a number of, preferably 1 to 2, individual doses to achieve the desired result. In the case of a parenteral treatment, similar or (in particular in the case of the intravenous administration of the active compounds) as a rule lower dosages can be used. The determination of the optimal dosage and manner of administration of the active compounds necessary in each case can be easily carried out by any person skilled in the art on the basis of his/her expert knowledge.

The invention further relates to a pharmaceutical preparation for the treatment of airway disorders, which in an individual dose (tablet, capsule, etc.) contains a reversible proton pump inhibitor as active compound in a dose of between 3 and 40, in particular 5 and 20, mg.

If the reversible proton pump inhibitors are to be employed for the treatment of airway disorders, the pharmaceutical preparations can also contain one or more pharmacologically active constituents of other pharmaceutical groups. Examples which may be mentioned are: tranquillizers (for example from the group consisting of the benzodiazepines, e.g. diazepam), spasmolytics (e.g. bietamiverine or camylofine), anticholinergics (e.g. oxyphencyclimine or phencarbamide), local anaesthetics (e.g. tetracaine or procaine), and optionally also enzymes, vitamins or amino acids.

In particular to be emphasized in this connection is the combination of the reversible proton pump inhibitors with other pharmaceuticals which are customarily employed for the treatment of airway disorders.

1. A method of treating an airway disorder in a patient comprising administering a therapeutically effective amount of a reversible proton pump inhibitor or a solvate, hydrate, salt, hydrate of a salt or solvate of a salt thereof, to a patient in need thereof.

2. (Canceled)

3. (Canceled)

4. (Canceled)

5. A pharmaceutical composition comprising a reversible proton pump inhibitor or a pharmaceutically acceptable solvate, hydrate, salt, hydrate of a salt or solvate of a salt thereof and a pharmaceutically acceptable excipient and/or vehicle.

6. A kit comprising a pharmaceutical composition according to claim 5 and a reference to the fact that it can be employed for the treatment of airway disorders.

7. (Canceled)

8. (Canceled)

9. The method according to claim 1, wherein the reversible proton pump inhibitor is selected from the group consisting of AU-461, Soraprazan (BY359), DBM-819, KR-60436, T-330, YH-1885, YJA-20379-8 and 2,3-dimethyl-8-(2-ethyl-6-methylbenzylamino)-imidazo(1,2-a)pyridine-6-carboxamide, or a pharmaceutically acceptable solvate, hydrate, salt, hydrate of a salt or solvate of a salt thereof.

10. The method according to claim 1, wherein the reversible proton pump inhibitor is selected from the group consisting of (7S,8R,9R)-2,3-dimethyl-7,8-dihydroxy-9-phenyl-7,8,9,10-tetrahydroimidazo[1,2-h][1,7]naphthyridine,
(7R,8R,9R)-3-hydroxymethyl-7,8-dihydroxy-2-methyl-9-phenyl-7,8,9,10-tetrahydroimidazo[1,2-h][1,7]naphthyridine,
(7S,8R,9R)-7,8-isopropylidenedioxy-2,3-dimethyl-9-phenyl-7,8,9,10-tetrahydroimidazo[1,2-h][1,7]naphthyridine,
7,8-dihydroxy-9-phenyl-2,3-dimethyl-7H-8,9-dihydropyrano[2,3-c]imidazo[1,2-a]pyridine,
(7R,8R,9R)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8R,9R)-2,3-dimethyl-8-hydroxy-7-methoxy-9-phenyl-7,8,9,10-tetrahydroimidazo[1,2-h][1,7]naphthyridine,
(7R,8S,9S)-2,3-dimethyl-8-hydroxy-7-methoxy-9-phenyl-7,8,9,10-tetrahydroimidazo[1,2-h][1,7]naphthyridine,
(7R,8R,9R)-2,3-dimethyl-7-ethoxy-8-hydroxy-9-phenyl-7,8,9,10-tetrahydroimidazo[1,2-h][1,7]naphthyridine,
(7S,8R,9R)-2,3-dimethyl-7-ethoxy-8-hydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7,8,9,10-tetrahydroimidazo[1,2-h][1,7]naphthyridine,
(7S,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-7-(2-methoxyethoxy)-9-phenyl-7,8,9,10-tetrahydroimidazo[1,2-h][1,7]naphthyridine,
(7R,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-9-phenyl-7-(2-propoxy)-7,8,9,10-tetrahydroimidazo[1,2-h][1,7]naphthyridine,
(7R,8R,9R)-2,3-dimethyl-7,8-dimethoxy-9-phenyl-7,8,9,10-tetrahydroimidazo[1,2-h][1,7]naphthyridine,
(7R,8R,9R)-2,3-dimethyl-8-hydroxy-7-(2-methylthioethyloxy)-9-phenyl-7,8,9,10-tetrahydroimidazo[1,2-h][1,7]naphthyrldine,
(7S,8R,9R)-2,3-dimethyl-8-hydroxy-7-(2-methylthioethyloxy)-9-phenyl-7,8,9,10-tetrahydroimidazo[1,2-h][1,7]naphthyridine,
(7R,8R,9R)-2,3-dimethyl-8-hydroxy-7-(2-methylsulphinylethoxy)-9-phenyl-7,8,9,10-tetrahydroimidazo[1,2-h][1,7]naphthyridine,
(7S,8R,9R)-2,3-dimethyl-8-hydroxy-7-(2-methylsulphinylethoxy)-9-phenyl-7,8,9,10-tetrahydroimidazo[1,2-h][1,7]naphthyridine,
(7R,8R,9R)-2,3-dimethyl-8-hydroxy-7-(ethylthio)-9-phenyl-7,8,9,10-tetrahydroimidazo[1,2-h][1,7]naphthyridine,
(7S,8R,9R)-2,3-dimethyl-8-hydroxy-7-(ethylthio)-9-phenyl-7,8,9,10-tetrahydroimidazo[1,2-h][1,7]naphthyridine,
(7R,8R,9R)-2,3-dimethyl-8-hydroxy-7-(2,2,2-trifluoroethoxy)-9-phenyl-7,8,9,10-tetrahydroimidazo[1,2-h][1,7]naphthyridine,
(7S,8R,9R)-2,3-dimethyl-8-hydroxy-7-(2,2,2-trifluoroethoxy)-9-phenyl-7,8,9,10-tetrahydroimidazo[1,2-h][1,7]naphthyridine,
(7S,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-methoxy-2,3-dimethyl-9-phenyl-7,8,9,10-tetrahydroimidazo[1,2-h][1,7]naphthyridine,
(7R,8R,9R)-8-acetoxy-7-ethoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-9-phenyl-8-propionyloxy-7,8,9,10-tetrahydroimidazo[1,2-h][1,7]naphthyridine,
(7R,8R,9R)-8-benzoyloxy-7-(2-methoxyethoxy)-2,3-dimethyl-9-phenyl-7,8,9,10-tetrahydroimidazo[1,2-h][1,7]naphthyridine,
(7S,8R,9R)-8-benzoyloxy-7-(2-methoxyethoxy)-2,3-dimethyl-9-phenyl-7,8,9,10-tetrahydroimidazo[1,2-h][1,7]naphthyridine,
(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-methoxy-2,3-dimethyl-9-phenyl-7,8,9,10-tetrahydroimidazo[1,2-h][1,7]naphthyridine,
(7S,8R,9R)-8-benzoyloxy-7-methoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine, (7S,8R,9R)-7-methoxy-2,3-dimethyl-8-(3-nitrobenzoyloxy)-9-phenyl-7,8,9,10-tetrahydroimidazo[1,2-h][1,7]naphthyridine,
(7R,8R,9R)-7-methoxy-2,3-dimethyl-8-(3-nitrobenzoyloxy)-9-phenyl-7,8,9,10-tetrahydrolmidazo[1,2-h][1,7]naphthyridine,
(7S,8R,9R)-7-(2-methoxyethoxy)-2,3-dimethyl-8-(4-methoxybenzoyloxy)-9-phenyl-7,8,9,10-tetrahydroimidazo [1,2-h][1,7]naphthyridine,
(7R,8R,9R)-7-(2-methoxyethoxy)-2,3-dimethyl-8-(4-methoxybenzoyloxy)-9-phenyl-7,8,9,10-tetrahydroimidazo [1,2-h][1,7]naphthyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-8-ethylaminocarbonyloxy-7-(2-methoxyethoxy)-2,3-dimethyl-9-phenyl-7,8,9,10-tetrahydroimidazo[1,2-h][1,7] naphthyridine,
(7R,8R,9R)-8-benzoyloxy-2,3-dimethyl-7-(2-methoxyethoxy)-9-phenyl-7H-8,9-dihydropyrano[2,3-c]imidazo[1,2-a]pyridine,
(7S,8R,9R)-8-benzoyloxy-2,3-dimethyl-7-(2-methoxyethoxy)-9-phenyl-7H-8,9-dihydropyrano[2,3-c]imidazo[1,2-a]pyridine,
(7R,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-2,3-dimethyl-7-methoxy-8-methoxyacetyloxy-9-phenyl-7,8,9,10-tetrahydroimidazo[1,2-h][1,7]naphthyridine,
(7R,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7S,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7R,8R,9R)-7-methoxy-8-methoxycarbonyloxy-2,3-dimethyl-9-phenyl-7,8,9,10-tetrahydroimidazo[1,2-h][1,7]naphthyridine,
(7S,8R,9R)-7-methoxy-8-methoxycarbonyloxy-2,3-dimethyl-9-phenyl-7,8,9,10-tetrahydroimidazo[1,2-h][1,7]naphthyridine,
(7R,8R,9R)-2,3-dimethyl-8-formyloxy-7-methoxy-9-phenyl-7,8,9,10-tetrahydroimidazo[1,2-h][1,7]naphthyridine,
(7S,8R,9R)-2,3-dimethyl-8-formyloxy-7-methoxy-9-phenyl-7,8,9,10-tetrahydroimidazo[1,2-h][1,7]naphthyridine,
(7R,8R,9R)-8-benzoyloxy-2,3-dimethyl-7-methoxy-9-phenyl-7,8,9,10-tetrahydroimidazo[1,2-h][1,7]naphthyridine,
(7R,8S,9R)-2,3,8-trimethyl-7,8-dihydroxy-9-phenyl-7,8,9,10-tetrahydroimidazo[1,2-h][1,7]naphthyridine,
(7S,8S,9R)-2,3-dimethyl-8-benzyl-7,8-dihydroxy-9-phenyl-7,8,9,10-tetrahydroimidazo[1,2-h][1,7]naphthyridine,
(7R,8S,9R)-2,3,8-trimethyl-7,8-0,0-isopropylidene-9-phenyl-7,8,9,10-tetrahydroimidazo[1,2-h][1,7]naphthyridine,
(7S,8S,9R)-2,3,8-trimethyl-7-(2-methoxyethoxy)-8-hydroxy-9-phenyl-7,8,9,10-tetrahydroimidazo[1,2-h][1,7]naphthyridine,
(7S,8S,9R)-2,3,8-trimethyl-7-methoxy-8-hydroxy-9-phenyl-7,8,9,10-tetrahydroimidazo[1,2-h][1,7]naphthyridine,
(7R,8R,9R)-2,3,7-trimethyl-7,8-dihydroxy-9-phenyl-7,8,9,10-tetrahydroimidazo[1,2-h][1,7]naphthyridine,
(7R,8R,9R)-2,3,7-trimethyl-7,8-[1,3]dioxolo-9-phenyl-7,8,9,10-tetrahydroimidazo[1,2-h][1,7]naphthyridine,
(8S,9R)-2,3-dimethyl-8-hydroxy-7-methylidene-9-phenyl-7,8,9,10-tetrahydroimidazo[1,2-h][1,7]naphthyridine,
(7S,8R,9R)-2,3,7-trimethyl-7,8-dihydroxy-9-phenyl-7H-8,9-dihydropyrano[2,3-c]imidazo[1,2-a]pyridine,
(7R,8R,9R)-2,3,7-trimethyl-7,8-dihydroxy-9-phenyl-7H-8,9-dihydropyrano[2,3-c]imidazo[1,2-a]pyridine,
(7S,8R,9R)-2,3-dimethyl-7,8-dihydroxy-7,9-diphenyl-7H-8,9-dihydropyrano[2,3-c]imidazo[1,2-a]pyridine,
(7S,8R,9R)-2,3-dimethyl-7-(21,2′-dimethylvinyl)-7,8-dihydroxy-9-phenyl-7H-8,9-dihydropyrano[2,3-c]imidazo [1,2-a]pyridine,
(7R,8R,9R)-2,3-dimethyl-7,8-O-isopropylidene-9-phenyl-7-vinyl-7H-8,9-dihydropyrano[2,3-c]-imidazo[1,2-a]pyridine,
(7R,8R,9R)-2,3-dimethyl-8-hydroxy-7-(2-methoxyethoxy)-9-phenyl-7H-8,9-dihydropyrano[2,3-c]imidazo[1,2-a]pyridine,
(7S,8R,9R)-2,3-dimethyl-8-hydroxy-7-(2-methoxyethoxy)-9-phenyl-7H-8,9-dihydropyrano[2,3-c]imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]pyridine,
(7S,8R,9R)-2,3-dimethyl-8-hydroxy-7-ethoxy-9-phenyl-7H-8,9-dihydropyrano[2,3-c]imidazo[1,2-a]pyridine,
(7R,8R,9R)-2,3-dimethyl-8-hydroxy-7-(2-methoxypropoxy)-9-phenyl-7H-8,9-dihydropyrano[2,3-c]imidazo[1,2-a]pyridine,
(7S,8R,9R)-2,3-dimethyl-8-hydroxy-7-(2-methoxypropoxy)-9-phenyl-7H-8,9-dihydropyrano[2,3-c]imidazo[1,2-a]pyridine,
(7R,8R,9R)-2,3-dimethyl-8-hydroxy-7-(2-propoxy)-9-phenyl-7H-8,9-dihydropyrano[2,3-c]imidazo[1,2-a]pyridine,
(7S,8R,9R)-2,3-dimethyl-8-hydroxy-7-(2-propoxy)-9-phenyl-7H-8,9-dihydropyrano[2,3-c]imidazo[1,2-a]pyridine,
(7R,8R,9R)-2,3-dimethyl-8-hydroxy-7-butoxy-9-phenyl-7H-8,9-dihydropyrano[2,3-c]imidazo[1,2-a]pyridine,
(7S,8R,9R)-2,3-dimethyl-8-hydroxy-7-butoxy-9-phenyl-7H-8,9 dihydropyrano[2,3-c]imidazo[1,2-a]pyridine,
(7S,8R,9R)-7,8-dihydroxy-6-methoxymethyl-2,3-dimethyl-9-phenyl-7,8,9,10-tetrahydroimidazo[1,2-h][1,7]naphthyridine,
(7R,8R,9R)-7,8-dihydroxy-6-methoxymethyl-2,3-dimethyl-9-phenyl-7,8,9,10-tetrahydroimidazo[1,2-h][1,7]naphthyridine,
(7S,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7S,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7R,8R,9R)-8-hydroxy-7-ethoxy-6-methoxymethyl-2,3-dimethyl-9-phenyl-7,8,9,10-tetrahydroimidazo[1,2-h][1,7]naphthyridine,
(7S,8R,9R)-8-hydroxy-7-ethoxy-6-methoxymethyl-2,3-dimethyl-9-phenyl-7,8,9,10-tetrahydroimidazo[1,2-h][1,7]naphthyridine,
7,8-dihydroxy-2,3-dimethyl-9-(3-thienyl)-7,8,9,10-tetrahydroimidazo[1,2-h][1,7]naphthyridine,
7-hydroxy-2,3-dimethyl-9-(3-thienyl)-7,8,9,10-tetrahydroimidazo[1,2-h][1,7]naphthyridine,
9-(3-furyl)-7-hydroxy-2,3-dimethyl-7,8,9,10-tetrahydroimidazo[1,2-h][1,7]naphthyridine,
(7R,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7S,8R,9R)-8-hydroxy-7-[2-(2-methoxyethoxy)ethoxy]-2,3-dimethyl-9-phenyl-7,8,9,10-tetrahydroimldazo[1,2-h][1,7]naphthyridine,
(7R,8R,9R)-7,8-dihydroxy-2-methyl-9-phenyl-7,8,9,10-tetrahydroimidazo[1,2-h][1,7]naphthyridine,
(7S,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-3-bromo-8-hydroxy-7-(2-methoxyethoxy)-2-methyl-9-phenyl-7,8,9,10-tetrahydroimidazo[1,2-h][1,7]naphthyridine,
(7R,8R,9R)-3-chloro-8-hydroxy-7-(2-methoxyethoxy)-2-methyl-9-phenyl-7,8,9,10-tetrahydroimidazo[1,2-h][1,7]naphthyridine,
(7R,8R,9R)-3-bromo-7-hydroxy-8-(2-methoxyethoxy)-2-methyl-9-phenyl-7,8,9,10-tetrahydroimidazo[1,2-h][1,7]naphthyridine,
(7R,8R,9R)-3-chloro-8-hydroxy-7-(2-methoxyethoxy)-2-methyl-9-phenyl-7H-8,9-dihydro-pyrano[2,3-c]imidazo[1,2-a]pyridine,
(7R,8R,9R)-8-hydroxy-7-(2-methoxyethoxy)₂-methyl-9-phenyl-7H-8,9-dihydro-pyrano[2,3-c]imidazo[1,2-a]pyridine,
(7R,8R,9R)-7,8-dihydroxy-2-methyl-9-phenyl-7H-8,9-dihydropyrano[2,3-c]imidazo[1,2-a]pyridine,
(7S,8R,9R)-7,8-dihydroxy-2-methyl-9-phenyl-7,8,9,10-tetrahydroimidazo[1,2-h][1,7]naphthyridine,
(7R,8R,9R)-8-hydroxy-7-methoxy-2-methyl-9-phenyl-7,8,9,10-tetrahydroimidazo[1,2-h][1,7]naphthyridine,
(7S,8R,9R)-8-hydroxy-7-methoxy-2-methyl-9-phenyl-7,8,9,10-tetrahydroimidazo[1,2-h][1,7]naphthyridine,
(7R,8R,9R)-3-hydroxymethyl-8-hydroxy-7-(2-methoxyethoxy)-2-methyl-9-phenyl-7,8,9,10-tetrahydroimidazo[1,2-h][1,7]naphthyridine,
(7R,8R,9R)-3-hydroxymethyl-8-hydroxy-7-(2-hydroxyethoxy)-2-methyl-9-phenyl-7,8,9,10-tetrahydroimidazo[1,2-h][1,7]naphthyridine,
(7R,8R,9R)-2,3-dimethyl-8-hydroxy-7-(2-hydroxyethoxy)-9-phenyl-7,8,9,10-tetrahydroimidazo[1,2-h][1,7]naphthyridine,
(7R,8R,9R)-3,9-diphenyl-8-hydroxy-7-(2-methoxyethoxy)-2-methyl-7,8,9,10-tetrahydroimidazo[1,2-h][1,7]naphthyridine,
(7R,8R,9R)-7,8-dihydroxy-2-methoxymethyl-3-methyl-9-phenyl-7,8,9,10-tetrahydroimidazo[1,2-h][1,7]naphthyridine,
(7S,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7S,8R,9R)-7-ethoxy-8-hydroxy-2-methoxymethyl-3-methyl-9-phenyl-7,8,9,10-tetrahydroimidazo[1,2-h][1,7]naphthyridine,
(7R,8R,9R)-7-ethoxy-8-hydroxy-2-methoxymethyl-3-methyl-9-phenyl-7,8,9,10-tetrahydroimidazo[1,2-h][1,7] naphthyridine,
and pharmaceutically acceptable solvates, hydrates, salts, hydrates of the salts and solvates of the salts thereof.

11. The method according to claim 1, wherein the reversible proton pump inhibitor is (7R,8R,9R)-2,3-dimethyl-8-hydroxy-7-(2-methoxyethoxy)-9-phenyl-7,8,9,10-tetrahydroimidazo[1,2-h][1,7]naphthyridine or a pharmaceutically acceptable solvate, hydrate, salt, hydrate of a salt or solvate of a salt thereof.

12. The method according to claim 1, wherein the airway disorder is chosen from bronchitis, COPD, asthma, pneumonitis or pulmonary fibrosis.
